(19) United States
(12) Patent Application Publication
(10) Pub. No.: US 2018/0149464 A1
(43) Pub. Date: May 31, 2018

(54) AUTOMATED OPTICAL COHERENCE TOMOGRAPHY SCANNING

(71) Applicant: Novartis AG, Basel (CH)

(72) Inventors: Mauricio Jochinsen, Fountain Valley, CA (US); Hugang Ren, Cypress, CA (US); Lingfeng Yu, Rancho Santa Margarita, CA (US)

(21) Appl. No.: 15/808,039

(22) Filed: Nov. 9, 2017

Related U.S. Application Data
(60) Provisional application No. 62/428,347, filed on Nov. 30, 2016.

(51) Int. Cl. G01B 9/02; A61B 3/10; A61B 3/00

(52) U.S. Cl. CPC G01B 9/02044; G01B 9/02091; G01B 9/02015; A61B 3/0025; A61B 3/0041; A61B 3/0033; A61B 3/102

(57) ABSTRACT

An optical coherence tomography (OCT) system and method for scanning a defined area according to a pre-set or user-specified scanning pattern, the defined area surrounding a specified starting position or offset from the starting position. Such OCT systems and methods may be used to generate a pictorial representation of internal target structures the OCT sample beam passed through.

### TECHNICAL FIELD

This disclosure relates to optical coherence tomography (OCT), and more specifically, to systems and methods for automated OCT scanning.

### BACKGROUND

Surgery often involves precise removal of tissue or placement of incisions. Various surgical procedures require highly precise targeting of tissues or structures below the surface to ensure the operation is successful and to cause minimal damage to nearby tissue and structures. In ophthalmic surgery, in particular, visualization of internal structures below the surface of the eye is critical to planning and completing the procedure. In such situations, microscopes and other similar devices are insufficient to visualize the internal structures to the extent necessary to perform the procedure. One way to visualize tissues and structures deeper below the surface of the eye is through the use of OCT scanning. OCT scanning uses a beam of light to penetrate into the tissue, and a detector to detect light reflected back from the eye. The reflected light provides data relating to internal structures of the eye and surrounding tissues that the beam of light penetrating the tissue passed through.

### SUMMARY

The present disclosure provides a system for automated OCT scanning. The system includes an OCT imaging head coupled to a processor, the OCT imaging head operable to generate an OCT source beam, a reference mirror, a beam splitter operable to split the OCT source beam into a sample beam directed to a target and a reference beam directed to the reference mirror, a detector operable to detect an interference pattern of a reflected OCT beam, the reflected OCT beam containing a component reflected from the reference mirror and a component reflected from the target, and generate data relating to the interference pattern, an input device coupled to the processor and operable to specify a starting position or modify the starting position of the OCT source beam, a display operable to present a pictorial representation of internal target structures the sample beam passed through, and a processor configured to direct the OCT source beam to continuously scan a defined area, receive data from the detector relating to the interference pattern, process data relating to the interference pattern, generate a pictorial representation of internal target structures the sample beam passed through, using the data relating to the interference pattern, transmit the pictorial representation to the display.

In additional embodiments, which may be combined with one another unless clearly exclusive: the detector is a spectrophotometer; the input device is a coordinate input device, a tool tracking device, a joystick, or a touchscreen device; the input device is operable to specify or modify a defined area that is an irregular shape; the defined area to scan is an area adjacent to or offset from a starting position specified by the input device; the processor is further configured to direct the OCT source beam to continuously scan a defined area in a specified scanning pattern; the specified scanning pattern is a rectangular scanning pattern; the specified scanning pattern is a circular full circumference sweeping scanning pattern or a circular partial circumference sweeping scanning pattern; the processor is further configured to generate and transmit, and the display is further operable to present, the pictorial representation of internal target structures the sample beam passed through in real time; the pictorial representation of internal target structures the sample beam passed through is a three-dimensional image; the processor is further configured to generate and transmit, and the display is further operable to present, a pictorial representation that incorporates prior pictorial representations generated during the continuous scan to render a three-dimensional image; and the processor is further configured to generate and transmit, and the display is further operable to present the pictorial representation in real time.

The present disclosure further provides a method for performing automated OCT scanning. The method includes specifying a starting position to direct an OCT source beam, specifying a defined area for the OCT source beam to continuously scan, directing the OCT source beam to continuously scan the defined area, detecting an interference pattern of a reflected OCT beam by using a detector, the reflected OCT beam containing a component reflected from the reference mirror and a component reflected from a target, and generating data relating to the interference pattern, receiving data from the detector relating to the interference pattern, processing the data relating to the interference pattern to generate a pictorial representation of internal target structures a sample beam component of the OCT source beam passed through, and transmitting the pictorial representation to a display.

In additional embodiments, which may be combined with one another unless clearly exclusive: the defined area is continuously scanned in a specified scanning pattern; the specified scanning pattern is a rectangular scanning pattern, a partial circumference circular scanning pattern, or a full circumference circular scanning pattern; the pictorial representation of internal target structures the sample beam passed through is presented in real time; the pictorial representation of internal target structures the sample beam passed through is a three-dimensional image; and the pictorial representation incorporates prior pictorial representations generated during the continuous scan to render a three-dimensional image.

The above systems may be used with the above methods and vice versa. In addition, any system described herein may be used with any method described herein and vice versa.

### BRIEF DESCRIPTION OF THE DRAWINGS

For a more complete understanding of the present invention and its features and advantages, reference is now made to the following description, taken in conjunction with the accompanying drawings, which are not to scale, in which like numerals refer to like features, and in which:

FIG. 1 is a schematic representation of a system for automated OCT scanning;

FIG. 2A is a schematic representation of a two-dimensional (“2D”) OCT line B-Scan;

FIG. 2B is a digitally processed image of a 2D OCT line B-Scan;

FIG. 3 is a schematic representation of an automated OCT line B-Scan;

FIG. 4A is a schematic representation of an OCT rectangular sweeping scan;

FIG. 4B is a schematic representation of an OCT circular full circumference sweeping scan;

FIG. 4C is a schematic representation of an OCT circular partial circumference sweeping scan; and

FIG. 5 is a flow chart of a method of automated OCT scanning.

### DETAILED DESCRIPTION

In the following description, details are set forth by way of example to facilitate discussion of the disclosed subject matter. It should be apparent to a person of ordinary skill in the field, however, that the disclosed embodiments are exemplary and not exhaustive of all possible embodiments.

OCT is an interferometric analysis technique for structural examination of a sample that is at least partially reflective to light. The sample may be called a “target.” In OCT scanning, an OCT imaging head produces an OCT light beam (an “OCT source beam”) that is directed toward a beam splitter. The beam splitter splits the source beam into one beam directed at a reference mirror (the “reference beam”) and one beam directed at a sample material (the “sample beam”). When the reference beam is reflected from the reference mirror and the sample beam is reflected from the sample, the two reflected beams are recombined (forming a “reflected OCT beam”), and directed toward a detector. When recombined, the reflected beam from the sample interferes with the reflected beam from the reference mirror. This generates an interference pattern.

Sample characteristics may be determined by analysis of such interference patterns. An interference pattern may be processed to generate an electronic OCT image of the sample. The electronic OCT image is then presented on a display. The sample may be a tissue and the image may be of the tissue. The sample may be a biological tissue, such as a human eye. OCT techniques may image fine structures in a human eye to assist in diagnosis of an opthalmological health condition, development of a suitable treatment plan, and performance of a surgical procedure. The OCT source beam may be supplied in pulses, sweeping wavelengths, or a broad band light.

The electronic OCT image of the sample, such as a tissue, is presented on a display in any of a variety of images, such as in a one-dimensional (“1D”) image, such as an A-Scan image, a 2D image, such as a B-Scan image, or a three-dimensional (“3D”) volume.

An A-scan image is a 1D image of the OCT light scattering profile of tissue as a function of depth into the tissue roughly parallel to the sample beam. A-Scan images can be used to generate a B-Scan image and a 3D volume data set. A B-Scan image is a 2D cross-sectional image of tissue obtained by laterally combining a series of A-Scan images. Alternatively, a B-Scan image can be obtained from a 3D volume data set.

Each B-Scan image corresponds to a line B-Scan. A line B-Scan is a cross-sectional scan created by moving the OCT source beam in a linear direction, along the cross-section. For each line B-Scan, the user specifies the starting position of the OCT source beam. Depending on the clinical application of a B-Scan image, each line B-Scan across a cross-section of tissue may have the same or a different size, length, width, and shape. For example, a first line B-Scan of a tissue may be 1 millimeter (mm) long, and another line B-Scan of the same tissue may be 16 mm long. Line B-Scans may be arranged in any pattern. For example, line B-Scans may be arranged parallel to each other, they may be arranged in a radius from a common crossing point to create the image of a circular area, or they may be arranged as a rectangular raster scan. A collection of consecutive B-Scan images can be used to construct a 3D volume image.

In ophthalmic surgery, for example, a user may have interest in visualizing a broad area instead of a specific position or cross-section. The present disclosure provides a system for automated OCT scanning in which a scanning pattern for a defined area is implemented to direct the OCT source beam to continuously perform a line B-scan in the defined area without intervening user input. The system provides an input device for the user to specify a starting position from which a scanning pattern for the OCT source beam is initiated. Once the scanning pattern, which may be pre-set or specified by a user via an input device, is initialized, the OCT source beam performs automated line B-scanning within the defined area. The defined area may be of any shape, for example a circle, rectangle, or square surrounding or near an area of interest of an eye. The defined area may be a pre-set size or shape, or may be any user-specified size or shape. The two-dimensional B-Scans are collected across time and combined to provide a pseudo-3D OCT image display in real time. Real time may mean in less than half a second, in less than one second, or otherwise in less than the normal reaction time of a user of the visual information.

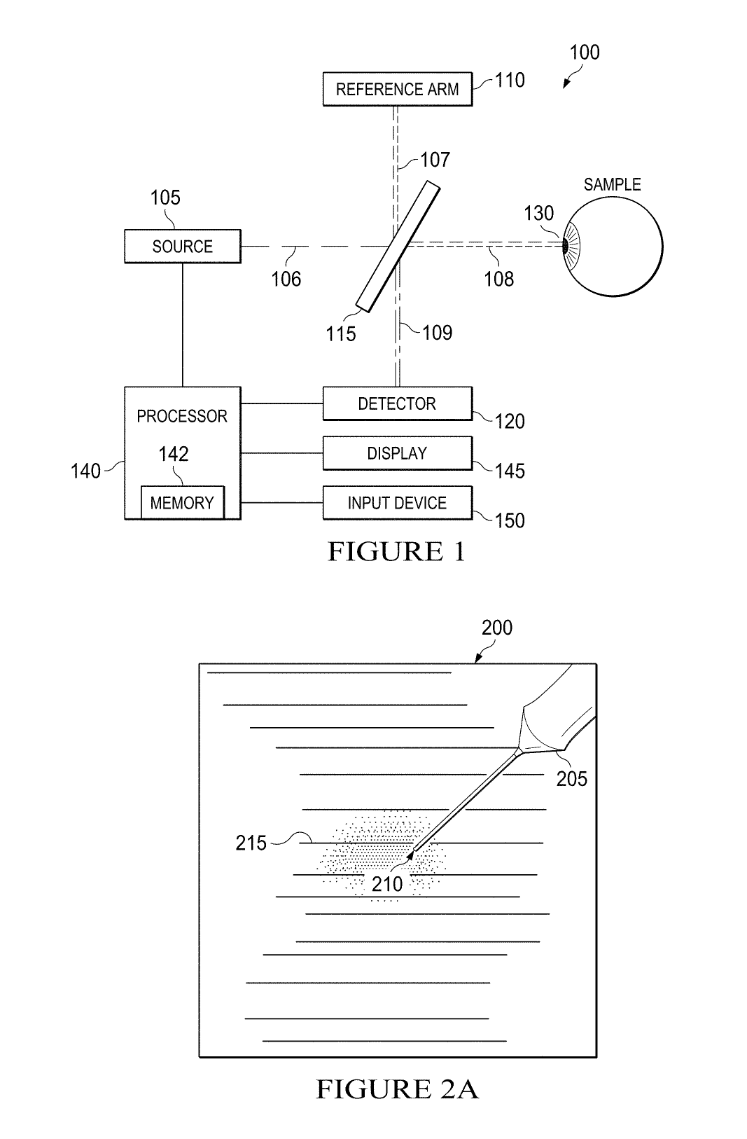

Referring now to the drawings, FIG. 1 is a schematic representation of a system 100 for automated OCT scanning. OCT system 100 includes OCT imaging head 105, which produces an OCT source beam that travels to beam splitter 115 where it is split so that the reference beam travels to reference mirror 110 and the sample beam travels to sample 130. Upon contacting the reference mirror 110 and sample 130, each beam is reflected. The reflected reference beam and the reflected sample beam each travel back to beam splitter 115, where they are recombined, creating an interference pattern. Detector 120 detects the interference pattern and sends a signal to processor 140, the signal containing data relating to the interference pattern. System 100 also provides input device 150 coupled to processor 140. Input device 150 is can be used to specify a starting position or modify a starting position of the OCT source beam. Input device 150 is further useable to specify a scanning pattern, which may be a pre-set pattern, modify a scanning pattern that is user-specified or pre-set, initialize, or cancel a scanning pattern. When a scanning pattern is initialized, processor 140 may direct the OCT source beam to perform automated line B-scanning in a defined area, according to the scanning pattern. Processor 140 uses the data to generate and transmit a pictorial representation relating to internal target structures the sample beam passed through. System 100 also provides display 145, which receives and displays the pictorial representation generated by processor 140.

As shown in FIG. 1, OCT imaging head 105 produces an OCT source beam 106 that travels to beam splitter 115, which splits OCT source beam 106 into reference beam 107 (directed at reference mirror 110) and sample beam 108 (directed at sample 130). Reference mirror 110 is positioned at a known distance from the OCT imaging head. Sample 130 may be an eye tissue. After reference beam 107 reaches reference mirror 110, it is reflected back toward beam splitter 115. Likewise, after sample beam 108 reaches sample 130, it is reflected back toward beam splitter 115. Both the reflected reference beam and the reflected sample beam are recombined at beam splitter 115 to form reflected OCT beam 109, which creates an interference pattern. Detector 120 may detect and transmit data to processor 140 relating to the interference pattern of reflected OCT beam 109. Detector 120 may be a spectrometer. Alternatively, detector 120 may include a photodiode or similar device that generates an electrical signal indicative of incident light intensity at detector 120. Detector 120 sends a signal, which may be electrical or wireless, to processor 140.

The user may specify the point at which sample beam 108 contacts sample 130 by controlling the starting position of OCT source beam 106. System 100 provides input device 150, which may be used to specify a starting position of the OCT source beam, modify the starting position, specify a defined area to scan, or modify the defined area to scan, the defined area specified in relation to the starting position of the OCT source beam. Input device 150 may be any input device, for instance, a joystick, a coordinate input device, a tool tracking device, or a touchscreen device. Input device 150 may be one or multiple input devices that may be coupled to each other and in communication with processor 140. Input device 150 may be further used to initialize, cancel, specify or modify a scanning pattern. When a scanning pattern is initialized, processor 140 may direct the OCT source beam to perform an automated scan within the defined area, according to the scanning pattern specified. The defined area may be of any shape, preferably relating to an area of interest of the eye. Input device 150 may be used to specify or modify a defined area that is a regular or an irregular shape. For example, a coordinate input device may be used to specify a circle or rectangle as the defined area, or a touchscreen device may be used to draw an irregular shape as the defined area to scan. The defined area may be an area surrounding the starting position. The defined area to scan may also be an area adjacent to or offset from the starting position. For example, the defined area may be a circle of a particular diameter surrounding the starting position of the OCT source beam. In another example, the defined area may be a circle of a particular diameter, but with the center point of the circle being adjacent to or offset from the starting position of the OCT source beam.

Once a starting position for the OCT source beam and a defined area to scan are specified via input device 150, a scanning pattern may be initiated via input device 150. Processor 140, which is coupled to OCT imaging head 105, may direct the OCT source beam to perform an automated scan according to the scanning pattern. The scanning pattern may be a pre-set scanning pattern or any scanning pattern specified by the user, for example, a rectangular sweeping scan, a circular partial circumference sweeping scan, or a circular full circumference sweeping scan. Processor 140 may direct the OCT source beam to perform the automated scanning pattern for any duration of time, for example, throughout the duration of surgery. As OCT imaging head 105 performs the specified scanning pattern, processor 140 may use the data received from detector 120, relating to the interference pattern, to generate a pictorial representation of the of internal target structures the sample beam passed through. The pictorial representation may be in the form of a 2D B-Scan image. Processor 140 may present subsequently generated 2D B-Scan images in real time. Processor 140 may also combine multiple 2D B-Scan images collected over time to provide a pseudo 3D OCT image display in real time.

The pictorial representation generated by processor 140 may be transmitted to display 145 and presented to the user. Display 145 may be configured to present such pictorial representations with display persistence. Display persistence, which may also be referred to as image persistence, is characterized by a display image that fades with time and is replaced or overwritten by a subsequently generated image. Even if the previous display image is not replaced or overwritten by a subsequently generated image, it still fades away. For example, a display may present a 2D B-Scan image and replace portions of the image with a subsequently generated 2D B-Scan image, in a manner similar to a airport radar display. In this example, the first 2D scan image fades and is gradually replaced by a second 2D scan image in a clockwise or counterclockwise manner. In another example, display persistence may be enabled using 3D scan images. Aspects of display persistence may be controlled by the user. For example, replacement of a presented image with a subsequently generated image may be paused, a previously-presented image may be recalled, and the refresh rate or “persistence rate” of replacing a presented image may be varied. The persistence rate may be defined as the rate at which a subsequently generated image replaces a previously displayed image, and may be adjusted from zero to any duration of time. For example, the persistence rate may be selected as 0.2 seconds.

A processor 140 may include, for example a microprocessor, microcontroller, digital signal processor (DSP), application specific integrated circuit (ASIC), field-programmable gate array (FPGA), or any other digital or analog circuitry configured to interpret and/or execute program instructions and/or process data. In some embodiments, processor 140 may interpret and/or execute program instructions and/or process data stored in memory 142. Memory 142 may be configured in part or whole as application memory, system memory, or both. Memory 142 may include any system, device, or apparatus configured to hold and/or house one or more memory modules. Each memory module may include any system, device or apparatus configured to retain program instructions and/or data for a period of time (e.g., computer-readable media). The various servers, electronic devices, or other machines described may contain one or more similar such processors or memories for storing and executing program instructions for carrying out the functionality of the associated machine.

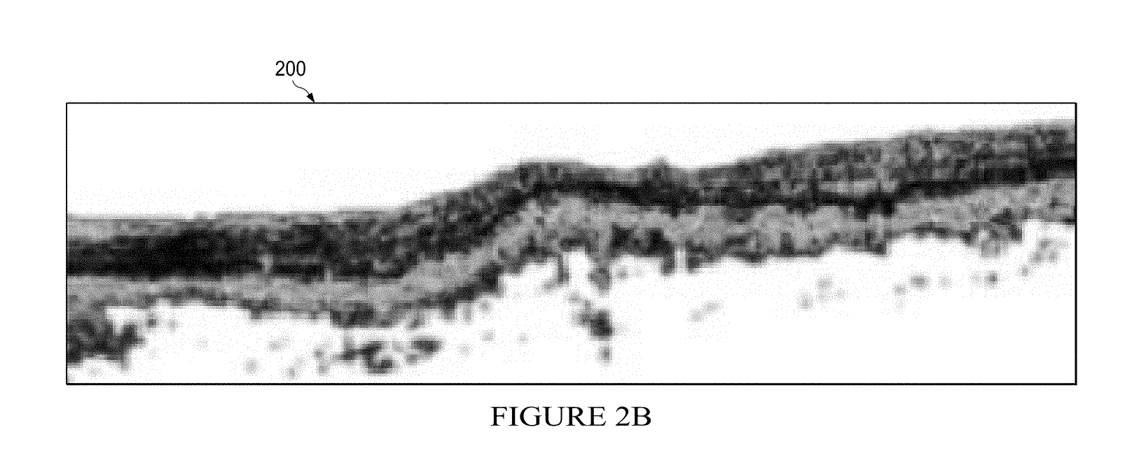

FIG. 2A is a schematic representation of an OCT line B-Scan. As illustrated in image 200, OCT source 205 directs its OCT source beam to starting position 210, which has been specified by the user via an input device. The OCT source beam performs the line B-Scan by directing its OCT source beam linearly along line 215. Once the line B-Scan is complete, the user must specify a new starting position via the input device to direct the OCT source beam to perform a subsequent line B-Scan.

FIG. 2B is a digitally processed image 200 of a 2D OCT line B-Scan. As illustrated, a line B-Scan may be performed to generate a pictorial representation relating to the internal target structures of the eye and surrounding tissues that the sample beam passed through.

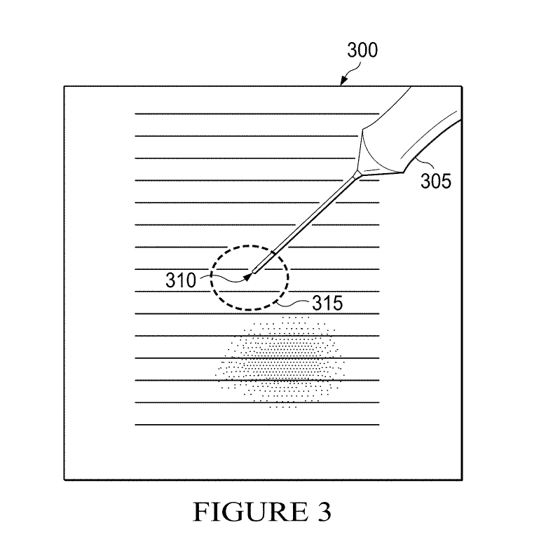

FIG. 3 is a schematic representation of an automated OCT line B-Scan. As illustrated in image 300, OCT imaging head 305 directs its OCT source beam to starting position 310, which has been specified by the user via an input device. Circular defined area 315 is also specified by the user via an input device. OCT imaging head 305 performs multiple automated line B-Scans within the defined area 315, without user intervention between each scan. In this example, the user may specify any scanning pattern to achieve an automated scan within circular defined area 315. OCT imaging head 305 may be configured to perform such automated scanning for any duration of time, such as throughout the duration of a surgical procedure.

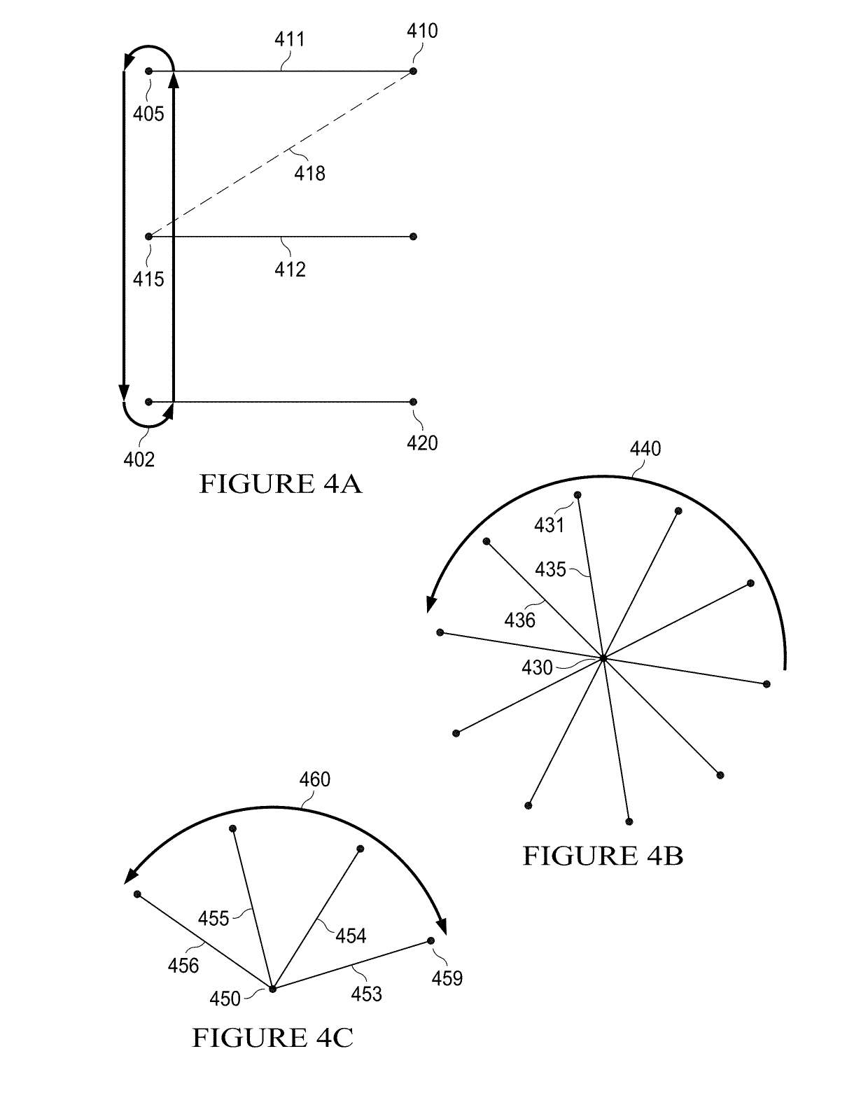

FIG. 4A is a schematic representation of an OCT rectangular sweeping scan, which may also be called a “raster” scan. As illustrated in FIG. 4A, a user may specify a starting position 405 for the OCT source beam, a defined area to scan, and here, a rectangular sweeping scan pattern. Once the scanning pattern is initiated, the OCT source beam may scan the defined area by directing the OCT source beam left to right along first line 411, beginning at starting position 405 and ending at end position 410. In this example, starting position 405 is in the top left corner of the defined area. At the end of line 411, the OCT source beam may pause or stop as it resets to second starting position 415, before beginning its scan of second line 412. Dotted line 418, connecting end position 410 of line 411 and starting position 415 of line 412, indicates the reset path of the OCT source beam between each line scan. This pattern of scanning left to right along each line and resetting, to the next line below the previous line, may continue without user intervention between each scan until the OCT source beam reaches position 420, which may be the bottom right border of the defined area. At this time, the OCT source beam may pause or stop and reset to starting position 405 and repeat this rectangular scanning pattern.

FIG. 4B is a schematic representation of an OCT circular full circumference sweeping scan. As illustrated in FIG. 4B, a user may specify a starting position 430 for the OCT source beam, a defined area to scan, and here, a circular full circumference sweeping scan. The OCT source beam may scan the defined area in a circular pattern moving from starting position 430 (in this example, the center point of the defined area) radially outward along line 435. At the end of line 435, indicated by end position 431, the OCT source beam may pause or stop as it resets to starting position 430. The OCT source beam then scans the next line 436 by beginning at starting position 430 and moving radially outward along line 436. This pattern of scanning each line from starting position 430 and moving radially outward to the end of each line may repeat continuously, without user intervention between each line scan. This pattern may repeat throughout any duration as specified by the user and may repeat in a clockwise or counterclockwise rotation. In this example, arrow 440 indicates that the specified full circumference scan continues in a counterclockwise rotation.

FIG. 4C is a schematic representation of an OCT circular partial circumference sweeping scan. As illustrated in FIG. 4C, a user may specify a starting position 450 for the OCT source beam, a defined area to scan, and here, a circular sweeping scan pattern that is a partial circumference scan. The OCT source beam may scan the defined area in a circular pattern moving from starting position 450 (in this example, the center point of the defined area) radially outward along line 453. At the end of line 435, indicated by end position 459, the OCT source beam may pause or stop as it resets to starting position 450. The OCT source beam then scans the next line 454 by beginning at starting position 450 and moving radially outward along line 454. This pattern of scanning each line from starting position 450 and moving radially outward to the end of each line may repeat continuously, without user intervention between each line scan. In this example, lines 453 and 456 define the boundaries of the partial circumference. Thus, the OCT source beam may scan line 453 and each line in a counterclockwise rotation until it completes its scan of line 456. In this rotation, the OCT source beam will scan line 453, then line 454, then line 455, and finally line 456. When it completes its scan of line 456, the OCT source beam will reset to starting position 450 and proceed to scan each line in a clockwise rotation until it completes its scan of line 453. In this rotation, the OCT source beam will scan line 456, then line 455, then line 454, and finally line 453. This pattern may repeat throughout any duration as specified by the user and may repeat in a counterclockwise then clockwise rotation, or a clockwise then counterclockwise rotation, as indicated by arrow 460.

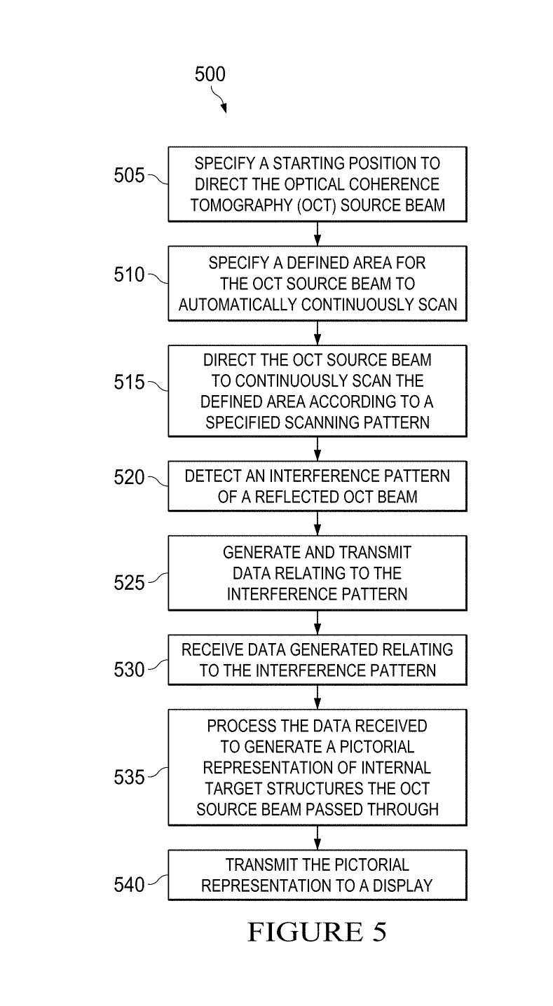

FIG. 5 is a flow chart of a method of automated OCT scanning. At step 505, a user may specify a starting position to direct the OCT source beam. The starting position may also be used as a reference position, from which the actual starting position of a scan may be defined as adjacent to or offset from such reference position. At step 510, a user may specify a defined area (the “defined area”) for the OCT source beam to continuously scan. This defined area may be of any shape, for example a circle, rectangle, or square surrounding or near an area of interest of an eye. The defined area may be a regular or an irregular shape. At step 515, the OCT source beam may be directed to continuously scan the defined area according to a specified scanning pattern. The scanning pattern specified may be any scanning pattern, for example, a rectangular sweeping scan, a circular full circumference sweeping, or a circular partial circumference sweeping scan. The scanning pattern may be configured to direct the OCT source beam to continuously scan the defined area without intervening user input for any duration, for example, throughout the duration of a surgical procedure.

At step 520, an interference pattern of the reflected OCT beam is detected by a detector. The reflected OCT beam includes the recombined beams reflected from the sample and the reference mirror. At step 525, data relating to the interference pattern may be generated and transmitted, the data indicating the internal target structures the sample beam passed through. At step 530, data relating to the interference pattern may be received and processed at step 535 to generate a pictorial representation of the internal target structures the sample beam passed through. At step 540, the pictorial representation may be transmitted to a display and may be presented to a user, for example, during a surgical procedure.

Such pictorial representations may be displayed continuously in real time or may be displayed sequentially, as directed by the user. For example, each pictorial representation may be displayed and continuously replaced with the next pictorial representation generated in real time. In another example, each pictorial representation may be displayed but only replaced with the next pictorial representation generated upon user confirmation (such as by pressing a button to provide confirmation on an input device).

The pictorial representations may also be displayed with display persistence. As described in FIG. 1, display persistence is characterized by a display image that fades with time and is replaced by or overwritten with a subsequently generated image. Even if the previous display image is not replaced or overwritten by a subsequently generated image, it still fades away. For example, a display may present a first pictorial representation and replace portions of it with a subsequently generated pictorial representation, in a manner similar to an airport radar display. In this example, the first pictorial representation fades and is gradually replaced by a second pictorial representation in a clockwise or counterclockwise manner. Aspects of display persistence may be controlled by the user. For example, replacement of a presented image with a subsequently generated image may be paused, a previously-presented image may be recalled, and the refresh rate or “persistence rate” of replacing a presented image may be varied. The persistence rate may be defined as the rate at which a subsequently generated image replaces a previously displayed image, and may be adjusted from zero to any duration of time. For example, the persistence rate may be selected as 0.2 seconds.

Method 500 may be implemented using the system of FIG. 1, or any other suitable system. The preferred initialization point for such methods and the order of their steps may depend on the implementation chosen. In some embodiments, some steps may be optionally omitted, repeated, or combined. In some embodiments, some steps of such methods may be executed in parallel with other steps. In certain embodiments, the methods may be implemented partially or fully in software embodied in computer-readable media.

For the purposes of this disclosure, computer-readable media may include any instrumentality or aggregation of instrumentalities that may retain data and/or instructions for a period of time. Computer-readable media may include, without limitation, storage media such as a direct access storage device (e.g., a hard disk drive or floppy disk), a sequential access storage device (e.g., a tape disk drive), compact disk, CD-ROM, DVD, random access memory (RAM), read-only memory (ROM), electrically erasable programmable read-only memory (EEPROM), and/or flash memory; as well as communications media such wires, optical fibers, and other electromagnetic and/or optical carriers; and/or any combination of the foregoing.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. An automated optical coherence tomography (OCT) system comprising:
an OCT imaging head coupled to a processor, the OCT imaging head operable to generate an OCT source beam;
a reference mirror;
a beam splitter operable to split the OCT source beam into a sample beam directed to a target and a reference beam directed to the reference mirror;
a detector operable to detect an interference pattern of a reflected OCT beam, the reflected OCT beam containing a component reflected from the reference mirror and a component reflected from the target, and generate data relating to the interference pattern;
an input device coupled to the processor and operable to specify a starting position or modify the starting position of the OCT source beam;
a display operable to present a pictorial representation of internal target structures the sample beam passed through; and
a processor configured to:
direct the OCT source beam to scan a defined area;
receive data from the detector relating to the interference pattern;
process data relating to the interference pattern;
generate a pictorial representation of internal target structures the sample beam passed through, using the data relating to the interference pattern;
transmit the pictorial representation to the display.

2. The OCT system of claim 1, wherein the detector is a spectrophotometer.

3. The OCT system of claim 1, wherein the input device is a coordinate input device.

4. The OCT system of claim 1, wherein the input device is a tool tracking device.

5. The OCT system of claim 1, wherein the input device is a joystick.

6. The OCT system of claim 1, wherein the input device is a touchscreen device.

7. The OCT system of claim 1, wherein the input device is further operable to specify or modify a defined area that is an irregular shape.

8. The OCT system of claim 1, wherein the defined area to scan is an area surrounding a starting position specified by the input device.

9. The OCT system of claim 1, wherein the defined area to scan is an area adjacent to or offset from a starting position specified by the input device.

10. The OCT system of claim 1, wherein the processor is further configured to direct the OCT source beam to scan a defined area in a specified scanning pattern.

11. The OCT system of claim 10, wherein the specified scanning pattern is a rectangular scanning pattern.

12. The OCT system of claim 11, wherein the specified scanning pattern is a circular full circumference sweeping scanning pattern or a circular partial circumference sweeping scanning pattern.

13. The OCT system of claim 1, wherein the processor is further configured to generate and transmit, and the display is further operable to present, the pictorial representation of internal target structures the sample beam passed through in real time.

14. The OCT system of claim 1, wherein the pictorial representation of internal target structures the sample beam passed through is a three-dimensional image.

15. The OCT system of claim 1, wherein the processor is further configured to generate and transmit, and the display is further operable to present, a pictorial representation that incorporates prior pictorial representations generated during the continuous scan to render a three-dimensional image.

16. The OCT system of claim 15, wherein the processor is further configured to generate and transmit, and the display is further operable to present the pictorial representation in real time.

17. A method for performing automated optical coherence tomography (OCT) scanning, comprising:
specifying a starting position to direct an OCT source beam;
specifying a defined area for the OCT source beam to scan;
directing the OCT source beam to scan the defined area;
detecting an interference pattern of a reflected OCT beam by using a detector, the reflected OCT beam containing a component reflected from the reference mirror and a component reflected from a target, and generating data relating to the interference pattern;
receiving data from the detector relating to the interference pattern;
processing the data relating to the interference pattern to generate a pictorial representation of internal target structures a sample beam component of the OCT source beam passed through; and
transmitting the pictorial representation to a display.

18. The method of claim 17, wherein the defined area is scanned in a specified scanning pattern.

19. The method of claim 18, wherein the specified scanning pattern is a rectangular scanning pattern.

20. The method of claim 18, wherein the specified scanning pattern is a circular full circumference sweeping scanning pattern or a circular partial circumference sweeping scanning pattern.

21. The method of claim 17, wherein the pictorial representation of internal target structures the sample beam passed through is generated and transmitted in real time.

22. The method of claim 17, wherein the generated and transmitted pictorial representation of internal target structures the sample beam passed through incorporates prior pictorial representations generated during the continuous scan to render a three-dimensional image.
